(19) United States
(12) Patent Application Publication
(10) Pub. No.: US 2017/0157336 A1
(43) Pub. Date: Jun. 8, 2017

(54) HYPODERMIC NEEDLE SYSTEM

(71) Applicant: XEND MEDICAL SYSTEMS, LLC, SALT LAKE CITY, UT (US)

(72) Inventors: Randall J. Olson, Salt Lake City, UT (US); Owen D. Brimhall, South Jordan, UT (US)

(73) Assignee: XEND MEDICAL SYSTEMS, LLC, SALT LAKE CITY, UT (US)

(21) Appl. No.: 15/367,001

(22) Filed: Dec. 1, 2016

Related U.S. Application Data
(60) Provisional application No. 62/262,287, filed on Dec. 2, 2015.

(51) Int. Cl. A61M 5/32; A61L 31/10; A61L 31/14

(52) U.S. Cl. CPC A61M 5/3286; A61M 5/329; A61L 31/14; A61L 31/10; A61M 2005/3201; A61M 2205/195; A61M 2205/584; A61M 2205/586; A61M 2205/0205; A61L 2400/18

(57) ABSTRACT

A needle hypodermic system and method for reducing the incidence of infections due to administering fluids by injection and/or for reducing the risk of surface contamination of sample fluids in humans or animals is provided. The hypodermic needle system may include a first, outer needle with a bore having an open distal end, and a second, inner needle or cannula having a closed distal end and an opening proximal to the distal end which can be rotated or otherwise advanced between open and closed positions. Additionally, the second, inner needle may have a rounded or blunted end and filler material may be used to prevent bacteria or other microbes from being carried by the needles.

### BACKGROUND OF THE INVENTION

The present invention relates to a hypodermic needle system and a method to reduce the incidence of infection or contamination caused when the needle is inserted into a human or animal. In some embodiments, the present invention relates to a hypodermic needle with a second needle or cannula disposed in the hollow channel of the first needle with the two structures working together to minimize the risk of contamination when injecting or withdrawing fluids from the body.

There is a variety of situations in which a needle is inserted into a human or animal. When most people think of the use of a hypodermic needle, they think of administering a compound to achieve a therapeutic effect in humans or animals. Injecting a drug into its site of action increases the potential effectiveness of the drug. However, each injection carries a small risk of infection to the patient. As the needle penetrates the skin, the needle can carry bacteria and other pathogens into the subdermal tissues or bloodstream of the patient. Infection due to injections can often be decreased by using proper aseptic technique, such as by swabbing the region to be injected with antibacterial solution. However, few technicians wait the required 90 seconds for the solution to take full effect and elimination of all skin bacteria is difficult and rarely occurs. Additionally, there are a variety of procedures in which a needle must be inserted into the body through a tissue which cannot be readily sterilized, such as injections which are made through anymucus membranes.

Because other methods of administering the drug may be less effective or not effective at all, however, it may be required to inject the drug anyway to effectively treat a particular disease or condition. This is often the case, for example, when treating eye diseases or conditions because the blood-ocular barrier keeps most drugs out of the eye, and because tolerable sterilizing solutions are not very effective on the conjunctival surface. Under normal circumstances, the blood-ocular barrier protects a human or animal by providing natural resistance against organisms invading the vitreous humor, the clear gel that fills the space between the lens and the retina of the eyeball. The immune response of a human or animal to an organism that is introduced into the vitreous humor is more limited than if the organism was present in other areas of the body. Thus, a medical procedure that disrupts the integrity of the globe of the eye, such as intravitreal injections to treat a disease or condition of the eye, can lead to infection and inflammation of the eye, i.e. endophthalmitis.

It has been found that when the needle passes through the exterior membranes surrounding the eyeball (the conjunctiva), bacteria, which are present normally, can be introduced into the interior hollow channel of the needle and ultimately deposited in the vitreous humor when a substance is injected.

Moreover, because the conjunctiva cannot be readily sterilized prior to intravitreal injections the risk of infection is not as minimal as is desired with such injections. Some common complications of endophthalmitis are decreased vision and/or permanent vision loss. Some patients may even require enucleation (removal of the entire eye) to eradicate a blind and painful eye.

Intravitreal injection of various drugs has become a mainstay of treatment in ophthalmology. It is currently estimated that approximately 1000 to 3000 infections due to intravitreal injection occur each year with approximately half of those infections resulting in legal blindness. The number of injections given each year is increasing as the understanding of how to treat certain eye diseases or conditions increases, and/or new drugs for treating such diseases or conditions become available. For example, intravitreal injections may be given to treat viral retinitis, age-related macular degeneration, cystoid macular edema, diabetic retinopathy, uveitis, vascular occlusions, and even endophthalmitis. For the most common condition, wet macular degeneration, injections may have to be given monthly for the rest of the patient's life so that the cumulative risk of infection becomes substantial.

Even during injections involving areas other than the eye, infection and contamination are concerns. Needle bores have been shown to collect bacterial contamination during the puncturing process. In a person with a compromised immune system, the bacteria or other microbes can be carried into the body and cause infections.

When administering a pharmaceutical compound or other therapeutic agent to a human or animal by injection, or when inserting a needle to take a blood sample, the tissue surface that is to be penetrated by the needle is typically sterilized with a chemical, such as alcohol, iodine, etc. In some areas of the body, such as a mucous membrane like the conjunctiva that covers the eye, some chemicals cannot be used or are difficult to use. In other areas, such as the skin, it is common for the person inserting the needle not to wait a sufficient amount of time for the antiseptic to kill all of the bacteria and many bacteria in sweat glands, hair follicles and the deeper epidermis usually survive even if the antiseptic is used appropriately. Thus, it is fairly easy for the needle to become contaminated.

The failure to properly sterilize the skin also raises the risk of false positives when taking blood or other bodily fluid samples, such as culturing joint effusions, to determine if there is a blood borne or local infection. This is at greatest risk when the bacteria enter the bore of the needle because the layers of skin typically mechanically clean the exterior of the needle as it advances through the tissue. The interior bore of a hypodermic needle, however, may simply become partially filled with bacteria and other microbes which can either be injected into the patient when administering medicine, or drawn back into the syringe when taking a blood or other bodily fluid sample. This is a very common clinical quandary.

The ability of contaminating microbes to be drawn back into the syringe is another major problem in medicine. Studies suggest that, depending on the medical facility, 0.6% to 6% of blood tests are contaminated by bacteria and other microbes which are not actually in the patient's blood stream. (Hall and Lyman, Updated Review of Blood Culture Contamination, Clinical Microbology Reviews, October 2006, pages 788-802.) This is significant because bacteremia (the presence of bacteria in the blood), besides potentially causing wise spread bacterial infection throughout the body, can cause the immune system to release chemicals which can lead to widespread inflammation. The inflammation may result in blood clotting and organ damage. In some cases, the patient suffers a dramatic drop in blood pressure (septic shock) and organs shut down causing death. Each year sepsis kills more than 258,000 Americans and it ranked by the Center for Disease Control as the ninth leading cause of disease related deaths. www.cdc.goc/spsis/basic/qa.html (Oct. 13, 2015).

Because of the potentially devastating effects of sepsis, hospitals take bacteremia and the like very seriously. Patients showing symptoms consistent with bacteria or other microbes in the blood have blood tests done to ensure that they are not suffering from a blood infection. If the blood tests confirm bacteria, etc., in the blood, the patient will usually be admitted or held as an in-patient for several extra days to address the infection.

While hypodermic needles are a necessary tool in drawing blood for such tests, they also cause a significant financial loss for hospitals. In the process of puncturing the skin, the bore of the needle will often trap bacteria from the skin inside the bore of the needle. When blood is then drawn through the needle, the bacteria mixes with the blood supply from the patient. When the blood is tested, a positive result for bacterial infection is received even though the patient does not actually have bacteremia or sepsis. Because hospitals often receive flat fee reimbursements and the rate of false positives is so high, having patients who are not suffering from sepsis remain in the hospital for additional days can collectively cost hospitals billions of dollars per year. The patients who must remain in the hospital due to these false-positive blood tests lose workdays, and are exposed to further infections in a hospital environment.

A number of efforts have been made to help differentiate false positives for bacteremia from actual infections. These include attempts to differentiate risk based on the organism detected, the number of blood cultures testing positive, and time to growth of the bacteria. (Hall and Lyman, Updated Review of Blood Culture Contamination, Clinical Microbiology Reviews, October 2006, pages 788-802). Each of these, however, is only predictive and runs the risk that a person actually suffering from bacteremia is inaccurately determined to be a false positive for an infection.

Thus, there is a need for a hypodermic needle system and method that substantially minimizes the transmission of bacteria and the like when a needle is advanced through the skin or other external tissues of a person or animal. It is desirable that such a hypodermic needle system is relatively easy to use.

### SUMMARY

It is an object of the present invention to provide a hypodermic needle system and method of use to reduce the incidence of iatrogenic infection and/or to reduce the rate of false positives in blood and other bodily fluid tests.

The present disclosure includes different devices, systems, methods and applications which can reduce the incidence of iatrogenic infection and/or reduce the rate of false positives in blood tests and are thus applications of a common inventive concept. It should be appreciated that various devices, systems, methods and applications will have some benefits and may lack other benefits which are present in different devices, systems, methods and applications described herein. Therefore, the teachings of the present disclosure and any actual or intended benefit of any embodiments should not be read into the claims unless expressly stated therein.

According to one aspect of the present disclosure, a hypodermic needle system may include a first, outer needle having a hollow bore and a second, inner needle or cannula disposed in the hollow bore of the first, outer needle. The second, inner needle or cannula may be shaped to selectively occlude the distal (patient) end of the first, outer needle while the first needle is advancing though the skin, and then be repositioned to allow the injection or withdrawing of fluid once the first, outer needle is located in a blood vessel. The second, inner needle or cannula may have a blunt, rounded or flattened end so that when the second, inner needle or cannula is exposed, there is less risk of the distal end lacerating the wall of a blood vessel.

According to another aspect of the present disclosure, the second, inner needle or cannula has a closed distal end. The closed distal end may be disposed at the distal end of the first needle so as to be substantially flush therewith and occlude the bore and thereby minimize the risk of bacteria getting into the bore.

The second, inner needle or cannula may have an opening adjacent its closed distal end which is offset from a long axis of the second, inner needle so that when the second, inner needle is in a first, insertion position, the opening is occluded by the first, outer needle of the hypodermic needle system and may substantially prevent bacteria, fungi, or other organisms/contaminates from entering the hollow bore or lumen of the second, inner needle or cannula. When the second, inner needle is rotated relative to the first needle, the opening of the second needle is repositioned relative to the first needle so as expose the opening and allows fluid to be injected or withdrawn through the hypodermic needle. Where the end of the second, inner needle or cannula has been rounded, blunted or flattened, the end exposed to the patient is not sharp, thereby preventing accidental laceration of the tissue immediately adjacent the needle.

According to another aspect of the present disclosure, the seam between the face of the first, outer needle and the closed distal end of the second, inner needle may be filled or the entire distal face of the needled may be coated with a material which prevents or substantially minimizes the risk of bacteria entering the seam. In one embodiment, a material such as poly(lactic-co-glycolic acid)(“PLGA”), may be used, which is resistant to the adherence of bacteria and the like.

According to another aspect of the disclosure, the hypodermic needle system may comprise an antibiotic, antifungal, or sterilizing compound disposed on the distal end.

These and other aspects of the present disclosure may be realized in various hypodermic needle systems and methods of use as shown and described in the following figures and related description.

### BRIEF DESCRIPTION OF THE DRAWINGS

Various embodiments of the present invention are shown and described in reference to the numbered drawings wherein:

FIG. 1 shows a cross-sectional side view of a hypodermic needle system in accordance with some aspects of the present disclosure in a first, closed position;

FIG. 2 shows a cross-sectional view of a hypodermic needle system of FIG. 1 with the hypodermic needle system in a second, open position;

FIG. 3 shows an alternate configuration of a hypodermic needle system in accordance with principles of the present disclosure wherein the second, inner needle is rounded, blunted or flattened and the distal face thereof is covered with a coating while in a first, closed position;

FIG. 4 shows the configuration of FIG. 3 in a second open position;

FIG. 5 shows a perspective view of a hypodermic needle system in a first, closed position;

FIG. 6 shows a perspective view of the hypodermic needle system of FIG. 5 with the needle in a second, open position;

FIG. 7 shows a fragmented, side cross-sectional view of a hypodermic needle system in a closed position;

FIG. 8 shows a fragmented, side cross-sectional view of the hypodermic needle system of FIG. 7 with the inner needle having been rotated and advanced;

FIG. 9 shows a fragmented, de cross-sectional view of a hypodermic needle system in a closed position;

FIG. 10 shows a fragmented, side cross-sectional view of a hypodermic needle system of FIG. 9 with the inner needle being advanced;

FIG. 11 shows a top view of a hypodermic needle system with the inner needle in a closed, retracted position;

FIG. 12 shows a top view of the hypodermic needle system of FIG. 11 with inner needle in an open, extended position;

FIG. 13 shows a top view of a hypodermic needle system with a rotatably advanceable inner needle in a first, closed position;

FIG. 14 shows a top view of the hypodermic needle system of FIG. 13 with the inner needle in a second, extended or open position;

FIG. 15 shows a side cross-sectional view of a first, outer needle and a side view of a second, inner needle in accordance with one aspect of the present disclosure;

FIG. 16 shows a top view of a hypodermic needle system which may incorporate the needle configuration of FIG. 15;

FIG. 17 shows a top view of an alternate configuration of a hypodermic needle system;

FIG. 18 shows a side view of the hypodermic needle system of FIG. 17 with wings drawn up for forming a handle for holding by a person using the hypodermic needle system;

FIG. 19 shows a close-up view of another configuration of a hypodermic needle system according to the present disclosure with the needle in a first, closed position;

FIG. 20 shows a close-up view of the hypodermic needle system of FIG. 19 in a second, open position;

FIG. 21 shows a perspective view of a hypodermic needle system as may be used with various configurations discussed herein;

FIG. 22 shows a close-up view of the needle and actuation mechanism in the first, closed position; and

FIG. 23 show a close-up view of the needle and actuation mechanism in a second, open position.

It will be appreciated that the drawings are illustrative and not limiting of the scope of the invention which is defined by the appended claims. The various elements of the invention accomplish various aspects and objects of the invention. It is appreciated that not every element of the invention can be clearly displayed in a single drawing, and as such not every drawing shows each element of the invention.

### DETAILED DESCRIPTION

The drawings will now be discussed in reference to the numerals provided therein so as to enable one skilled in the art to practice the present invention. The drawings and descriptions are exemplary of various aspects of the invention and are not intended to narrow the scope of the appended claims. It will be appreciated that the various aspects of the hypodermic needle systems discussed herein may be the same or different aspects of the needle systems may be used together. Different reference numerals may be used to describe similar structures in the various hypodermic needle systems for clarity purposes only.

The skilled artisan will understand, that the methods described below can be practiced without employing these specific details, or that they can be used for purposes other than those described herein. Indeed, they can be modified and can be used in conjunction with products and techniques known to those of skill in the art in light of the present disclosure. Furthermore, it will be appreciated that the drawings may show some aspects of the invention in isolation and the elements in one figure may be used in conjunction with elements shown in other figures.

Reference in the specification to “one configuration,” “one embodiment” “one aspect” or “a configuration,” “an embodiment” or “an aspect” means that a particular feature, structure, or characteristic described in connection with the configuration may be included in at least one configuration and not that any particular configuration is required to have a particular feature, structure or characteristic described herein unless set forth in the claims. The appearances of the phrase “in one configuration” or similar phrases in various places in the specification are not necessarily all referring to the same configuration, and may not necessarily limit the inclusion of a particular element of the invention to a single configuration, rather the element may be included in other or all configurations discussed herein. Thus it will be appreciated that the claims are not intended to be limited by the representative configurations shown herein. Rather, the various representative configurations are merely representative examples and are provided to help one of ordinary skill in the art to practice the inventive concepts claimed herein.

Furthermore, the described features, structures, or characteristics of embodiments of the present disclosure may be combined in any suitable manner in one or more embodiments. In the following description, numerous specific details may be provided, such as examples of products or manufacturing techniques that may be used, to provide a thorough understanding of embodiments of the invention. One skilled in the relevant art will recognize, however, that embodiments discussed in the disclosure may be practiced without one or more of the specific details, or with other methods, components, materials, and so forth. In other instances, well-known structures, materials, or operations may not be shown or described in detail to avoid obscuring aspects of the invention.

Before the present invention is disclosed and described in detail, it should be understood that the present invention is not limited to any particular structures, process steps, or materials discussed or disclosed herein. More specifically, the invention is defined by the terms set forth in the claims. It should also be understood that terminology contained herein is used for the purpose of describing particular aspects of the invention only and is not intended to limit the invention to the aspects or embodiments shown unless expressly indicated as such. Likewise, the discussion of any particular aspect of the invention is not to be understood as a requirement that such aspect is required to be present apart from an express inclusion of that aspect in the claims.

It should also be noted that, as used in this specification and the appended claims, singular forms such as “a,” “an,” and “the” may include the plural unless the context clearly dictates otherwise. Thus, for example, reference to “a bracket” may include an embodiment having one or more of such brackets, and reference to “the needle body” may include reference to one or more of such needle bodies.

As used herein, the term “substantially” refers to the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing the nearly all of the length of a lumen would be substantially enclosed, even if the distal end of the structure enclosing the lumen had a slit or channel formed along a portion thereof. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

Concentrations, amounts, proportions and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

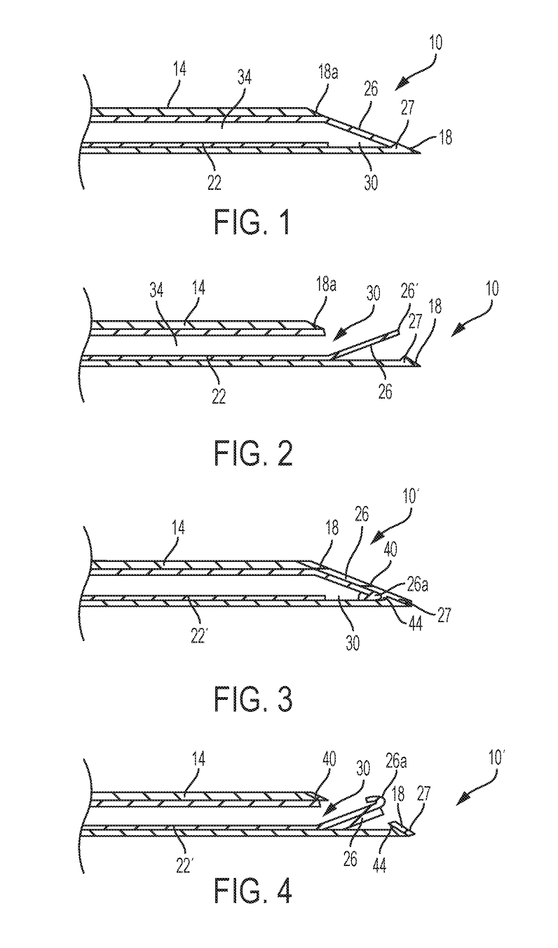

Turning to FIG. 1, there is shown a fragmented, cross-sectional view of a hypodermic needle system, generally indicated at 10, according to principles of the present invention. The hypodermic needle system 10 may include a first, outer needle 14 which is configured with a sharp face at the distal end 18 with a pointed or sharpened edge to be advanced into a person or animal to either inject a fluid into the person or animal or to withdraw fluid from the person or animal. As will be discussed below, the distal end may include a projection, rib or barb 27 on the inner surface of the needle at the distal end.

The hypodermic needle system 10 may also include a second, inner needle or cannula 22 disposed in the bore of the first, outer needle 14. The second, inner needle 22 has a closed distal end 26 that may be sized and shaped to generally align with the distal end 18 of the first, outer needle 14 so as to occlude the bore thereof. By filling the end of the bore in the outer needle 14, the closed distal end 26 prevents “coring” where there bore collects skin and bacteria and other pathogens. In a conventional needle, these contaminants can either infect the patient or can contaminate any samples obtained. (It will be appreciated in light of the present disclosure that the term needle relating to the second inner needle should be read broadly and may include cannula formed by materials other than metal). The distal end 26 may extend to the projection 27 on the interior of the first, outer needle as will be explained below.

The second, inner needle 22 may also include an opening 30 which is disposed adjacent to but behind the closed distal end 26. The opening 30 communicates with a hollow bore 34 in the second needle 22. As shown in FIG. 1, the second needle 22 is in a first position wherein the opening 30 is closed by the first, outer needle 14, so that the bore 34 of the second, inner needle 22 is not in communication with the patient.

The second, inner needle 22 may be rotatable relative to the first, outer needle 14 into a second, open position, as shown in FIG. 2. (It will be appreciated that the second inner needle 22 can be rotated within the first, outer needle 14, or the first, outer needle may be rotated about the second, inner needle, or both may be rotated relative to each other). Rotation of either of the needles 14 or 22 relative to the other leaves the opening 30 in an open position wherein it extends beyond the short side 18a of the distal end 18, thereby placing the bore 34 of the second, inner needle, in communication with the atmosphere or conditions outside the first, outer needle 14. Thus, for example, the hypodermic needle system 10 is advanced so that the first, outer needle 14 is inserted into a blood vessel or the like. Once the second, inner needle 22 is in the desired place, needles are then rotated relative to each other so that the needles are in the second, open position.

As the first, outer needle 14 is advanced through the skin, the skin mechanically wipes the exterior of the first, outer needle 14 and the closed end 26 of the second, inner needle 22, thereby removing bacteria and other pathogens. Once in the desired location, the needles are rotated into the second, open position and fluid can be injected and/or withdrawn through the bore 34 of the second, inner needle 22 without risk of contamination of the bore either infecting the patient or contaminating samples taken from the patient.

As was mentioned previously, the tip 26′ of the closed end 26 stops at the projection 27 on the inner surface of the first needle 14. If the closed end 26 matched the distal end 18 of the first needle, the tip 26′ of the closed end would be sharp can could lacerate a blood vessel when rotated into the position shown in FIG. 2. To prevent such, the tip 26′ is rounded, blunted, flatted etc., thereby reducing the risk of damage caused by the second inner needle rotating into position to inject or withdraw fluid into the body. The barb or projection 27 on the first, outer needle 22 helps prevent a gap being left between the tip 26′ of the second, inner needle 22 and the distal end 18 where bacterial may collect.

Turning now to FIG. 3 there is shown an alternate configuration of a hypodermic needle system 10′. Most of the structures of the hypodermic needle system 10′ are the same as those shown in FIGS. 1 and 2, and are therefore labeled accordingly. One difference between the two configurations relates to the second, inner needle 22′. Rather than having a blunted or flattened end on the closed end 26 of the second, inner needle 22 as shown in FIGS. 1 and 2, second inner needle 22′ has been formed with a rounded distal end 26a. The rounded distal end 26a makes it even less likely that the second, inner needle 22′ will cut a vessel or other tissue when it is moved into the second, open position. It will be appreciated that the distal tip 26′ or 26a may be formed by a variety of procedures including, but not limited to, molding, abrading, etching etc., to avoid leaving a sharp end which may inadvertently lacerate tissue.

Because of the rounded end 26a, it is difficult to make the closed end 26 of the second, inner needle 22′ closely align with the projection 27 on the distal end 18 of the first, outer needle 14. To further reduce the risk of bacteria or other pathogens being disposed on the distal end, a coating or film 40 may be disposed on the distal end 18 of the first, outer needle 14 and the closed end 26 of the second, inner needle 22′. The coating 40 may be applied in a liquid state so that is fills any voids (such as 44) along the seams between the first, outer needle 14 and the second, inner needle 22′. (It will be appreciated that the film or coating 40 could also be used on the embodiment of FIGS. 1 and 2, or any of the configurations described herein.)

The coating or film 40 may be comprised of biodegradable plastic, starch, PLGA, or any other microbial resistant, biodegradable, biocompatible material. For reasons which are discussed below, PLGA is a particularly beneficial material from which to make the coating 40. During dipping, the PLGA or other biocompatible material can be dissolved into an appropriate solvent, such as acetone. Various dilutions can be made by varying the ratio of PLGA or other biocompatible material to the solvent. Various viscosities can be made and optimized for the needle diameter and area to be covered and the thickness of the coating desired. Multiple dips can be used to build up material with appropriate drying times between dips. For sealing small gaps a thin, less viscous mix may be adequate.

During injection molding, a syringe or other applicator may be filled with the coating 40 solution. The solution may then be directly injected onto the distal end 18 and closed end 26 and allowed to cure to form a rigid or semi-rigid coating. The coating should be sufficiently thin, however, that it can be fractured by rotation of the needles and/or quickly dissolved by body fluids and the like. A thin coating may help reduce the incidence of micro-crevasses or irregularities which could harbor bacteria or other contamination. In addition, this method minimizes any blunting of the internal or external needle cutting edges, which might otherwise occur if thicker materials were used.

Use of the hypodermic needle system 10 of the present invention may be particularly beneficial when the first, outer needle 14 must be inserted into the body through a tissue which cannot be readily sterilized. The exterior of the needle 14 is mechanically cleaned by the tissue it passes through, thereby limiting the risk of infection. The bore of the needle 14 is protected by the closed end 26, which prevents bacteria and other microbes from getting into the bore.

While materials resistant to the adherence of bacteria and other micro-organisms are desirable to reduce contamination in either direction, materials such as PLGA are also beneficial in that they can be used to deliver medications. Thus, for example, a medication could be formed into the material which is used to form the coating. When the hypodermic needle system is disposed in a vein, the coating can dissolve, releasing the medication.

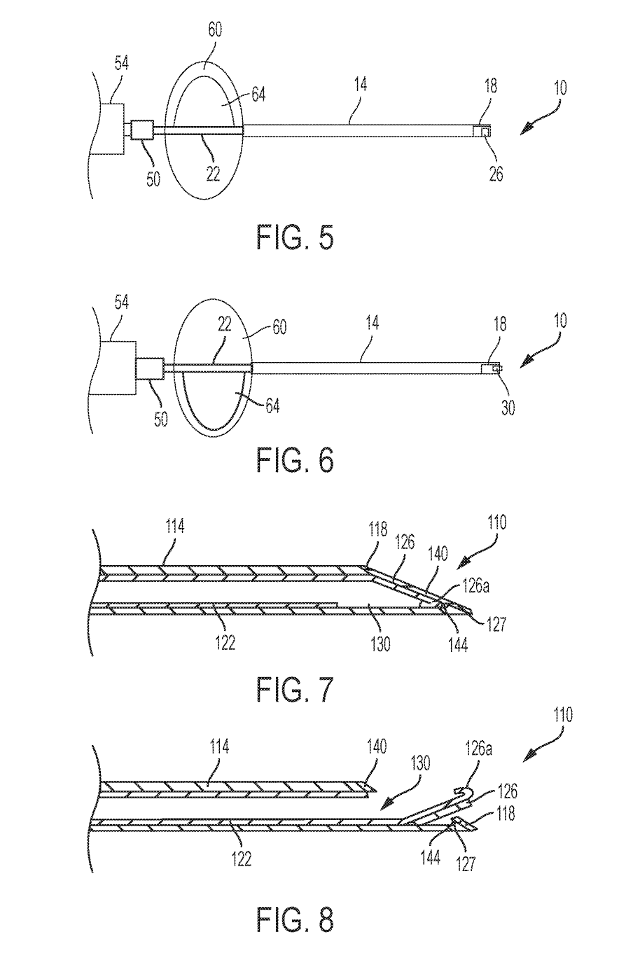

Turning now to FIGS. 5 and 6, there is shown a plan view of the hypodermic needle system 10. The second, inner needle 22 may extend proximally beyond the first, outer needle 14 and may be attached to an adaptor, such as a luer-lock 50, for attachment to a syringe 54. The syringe 54 may thus be used to inject fluid into or withdraw fluid from the second inner needle.

The first, outer needle 14 may be attached to a base 60, while the second, inner needle 22 is attached to an actuator 64, such as a lever, flange or handle. The actuator 64 is movable relative to the base 60 so as to rotate the second, inner needle 22 with respect to first, outer needle 14. Thus, FIG. 5 shows the two needles in the first, closed position as shown in FIGS. 1 and 3, while FIG. 6 shows the actuator 64 pivoted 180 degrees with respect to the base to thereby move the closed end 26 into the second, open position as shown in FIGS. 2 and 4. It will be appreciated that the base 60 could be numerous different structures including, but not limited to, a plate, a body, a box, or a strap. Likewise, the actuator could be numerous structures including, but not limited to, a flange, an arm, and a wheel.

One advantage of the structure shown is that it allows one-handed operation as the user may grab the base 60 and first, outer needle 14 and insert in into the patient's skin while in the first, closed orientation. Then a thumb or finger can then be used to rotate the actuator 64 to rotate the needles with respect to each other, and move the needles into the second, open configuration wherein the closed end 26 has been rotated so that the opening 30 connects the bore of the second, inner needle 22 to the tissue/bloodstream of the patient. The syringe 54 may then be actuated to inject or withdraw fluid which is much less likely to be contaminated than a conventional hypodermic needle.

Turning now to FIGS. 7 and 8 there is shown an alternate configuration of a hypodermic needle system 110. Most of the structures of the hypodermic needle system 110 are substantially the same as those discussed above with respect to FIGS. 1 through 4. One difference with the configuration shown in FIG. 7, however, is that the opening 130 is larger than the opening in the prior embodiments. In FIG. 7 the first, outer needle 114 may have a film 140 on its distal end 118 of the first, outer needle 114 and on the distal end 126 of the second, inner needle 122. This layer may cover any gap between the distal end 126 and the projection 127 if such is provided. It will also be appreciated that the film or other layer(s) of sealant material may be used to fill any void present due to a lack of a projection 127 on the inner surface of the first, outer needle. The film 140 covers the distal end 126 and the void(s) 144 which may exist between the ends of the first, outer needle and the second, inner needle. As the needle is advanced through tissue, the tissue will mechanically wipe bacteria and the like off the film 140.

The coating or film 140 may be comprised of biodegradable plastic, starch, PLGA, or any other microbial resistant, biodegradable, biocompatible material. For reasons which are discussed above, PLGA may be a particularly beneficial material from which to make the coating 140.

Rotating the second, inner needle 122 with respect to the first, outer needle 114 breaks, shears or otherwise disrupts the film 140 and exposes the opening 130 in the second, inner needle. The second, inner needle 122 may then be advanced within the first, outer needle 114 so that the opening 130, which is larger than opening 30 in FIG. 3, is more fully exposed, thereby facilitating the injection or withdrawal of fluid through the second, inner needle 122.

Because of the rounded end 126a, the risk that the second inner needle will puncture a vessel wall is reduced as the end of the second, inner needle 122 is advanced. Upon completion of the injection or withdrawal of fluid, the second, inner needle may be left in the location shown in FIG. 8, rotated back into the position shown in FIG. 7, or simply withdrawn back to the distal end of the first, outer needle to minimize the risk of the rounded end 126a catching on tissue.

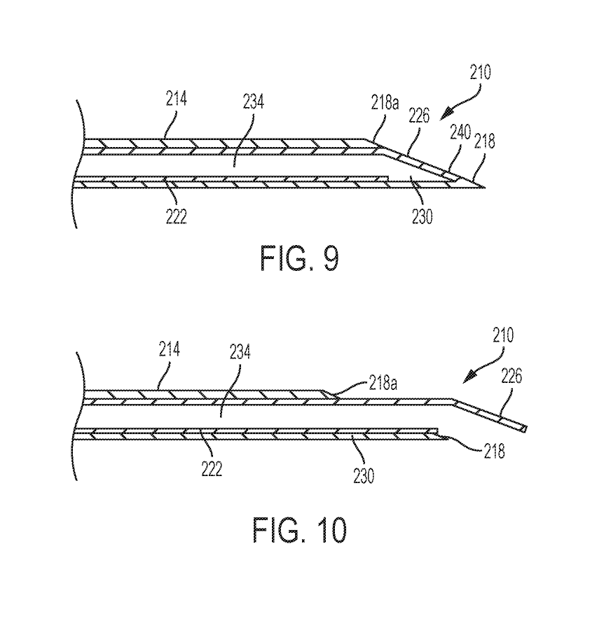

FIGS. 9 and 10 show fragmented, side cross-sectional views of an alternate embodiment of a hypodermic syringe system, generally indicated at 210. The syringe system 210 includes a first, outer needle 214 and a second, inner needle 222. The distal end of the second, inner needle 222 may have a closed end 226 which is beveled or angled to match a beveled distal end 218 of the first, outer needle 214. An opening 230 is formed by the second, inner needle 222 adjacent the distal end 226, with the opening being covered by the first, outer needle 214 when the second, inner needle is disposed in a first, closed position as shown in FIG. 9.

Rather than rotating the needle as discussed above with respect to FIGS. 1-8, the second, inner needle 222 may be slidable with respect to the first, outer needle 214 so as to expose the opening 230 formed by the second, inner needle as shown in FIG. 10. Thus, during use the hypodermic needle system 210 is inserted into a patient in the configuration shown in FIG. 9. Once the first, outer needle 214 is in the desired location, the second, inner needle 222 is advanced until a fluid can be passed through the bore 234 of the second, inner needle. The needles may then be withdrawn from the patient, or the second, inner needle 222 may be withdrawn back into the position shown in FIG. 9 prior to withdrawal of the first, outer needle 214. It will be appreciated that the distance to which the second, inner needle 222 may be advanced out of the first, outer needle 214 may depend on the desired used of the needle. For example, a needle system which is injecting a very low viscosity fluid into the body may require a very small opening 230, thereby only requiring minimal movement, while a needle system which is injecting a more viscous solution or is withdrawing blood may use a larger opening and thus require more movement by the second, inner needle 222.

While the distal tip of the second, inner needle's 222 distal end 226 may be sharp, it may also be flattened, rounded, or blunted as discussed above. As such, the risk of inadvertent injury is reduced. To minimize the risk of bacteria, etc., being carried on the face of the needle assembly, the distal ends 218 and 226 may be coated with a layer of film. In the alternative, filler material such as those discussed above may be placed between the distal tip of the distal end 226 and the interior surface of the first, outer needle adjacent the distal end 218 to prevent bacterial and other microbes from being trapped in the void left by the rounded tip, etc.

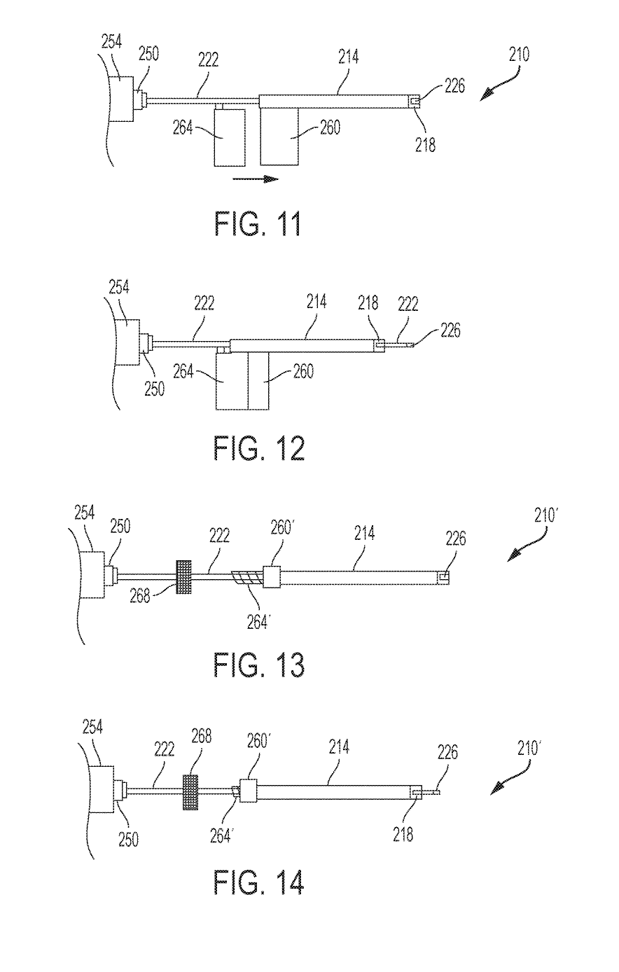

FIGS. 11 and 12 show a top view of a hypodermic needle assembly, such as that shown in FIGS. 9 and 10. In FIG. 11, the second, inner needle 222 is in a first, closed or retracted state. In such a configuration the distal end 226 of the second, inner needle may align with the distal end 218 of the first, outer needle 214 so as to minimize the risk of bacteria contaminating the bore of the first, outer needle.

The second, inner needle may have an adaptor 250, such as a luer lock, disposed on an opposite end for receiving a syringe body 254. When the adaptor 250 is attached to the syringe body 254, the syringe body may inject or withdraw fluid through the second, inner needle 222 when the second needle is in the second, open or extended position.

A flange or base 260 may be attached to the first, outer needle 214 and an actuator 264 may be attached to the second, inner needle 222. Advancing the actuator 264 toward the base 260 advances the second, inner needle 222 with respect to the first, outer needle 214, thereby moving the second, inner needle 222 into the second, open or extended position as shown in FIG. 12. Thus, in some configurations, medical or other personnel may insert the first, outer needle 214 into the skin of a person or animal with one hand, and then use the fingers of that hand to move the actuator 264 toward the base 260, thereby opening the hypodermic syringe system 210 for use. Because the distal end 226 of the second, inner needle 222 is aligned with the opening in the distal end 218 of the first, outer needle 214, the bore in the first, outer needle is blocked, thereby reducing bacterial contamination. Furthermore, bacteria coming into contact with the distal end 226 are likely to be mechanically wiped away prior to entering the blood stream. It will be appreciated that the actuator and the base may be varying distances apart depending on the size of the opening 230 desired.

Turning now to FIGS. 13 and 14, there is shown an alternate configuration of a hypodermic needle system 210′ configured for advancing the second, inner needle 222 relative the first, outer needle 214. Instead of sliding an actuator toward a base as shown in FIGS. 11 and 12, the hypodermic syringe system 210′ includes a base 260′ attached to the first, outer needle, and an actuator 264′ connected to the second, inner needle 222. As shown the actuator 264′ may be a threaded sleeve which engages threads (not show) of the base 260′ to advance or retract the second, inner needle 222 depending on the direction of rotation. A wheel 268 may be included as part of the actuator 264′. Rotating the wheel in one direction will rotate the threaded sleeve and cause the second, inner needle 220 to advance from the position shown in FIG. 13 to the position shown in FIG. 14. This moves the second, inner needle 222 from the first, retracted or closed position to the second, extended or open position wherein fluid can be injected or withdrawn through the opening (not shown) in the second, inner needle.

It will be appreciated that such a configuration may allow medical personnel and the like to insert the first, outer needle 214 into a vein or vessel, rotate the wheel 268 or otherwise rotate the second inner needle 222 to advance the needle until the opening is uncovered and then actuate a piston in the syringe body 254 to inject or withdraw fluid. All of this may be accomplished with one hand, such as, for example, the index finger and the thumb holding the first, outer needle 214, while the ring finger or the pinky is used to rotate the wheel and advance or retract the second, inner needle 222.

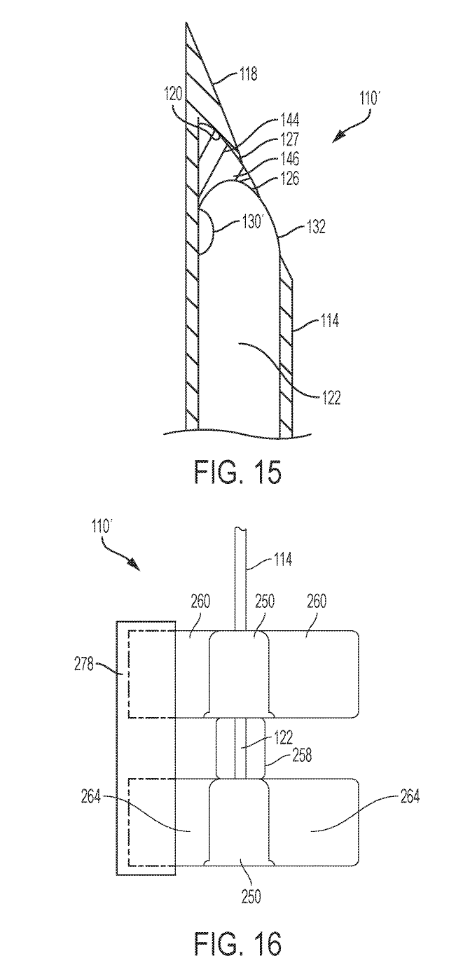

Turning now to FIG. 15, there is shown a side, partial cross-sectional view of a hypodermic needle system, generally indicated at 110′. The configuration is similar in many respects to that of FIGS. 7 and 8, and is therefore numbered accordingly. Specifically, there is a first, outer needle 114 and a second, inner needle or cannula 122′. The second, inner needle or cannula 122′ has a generally or broadly rounded distal end 126′ an opening 130 disposed adjacent to the distal end. For example, the radius of curvature at the broadly rounded distal end may be about 0.5 mm or less. It will be appreciated as used herein that the term non-sharp, rounded, flattened or blunted means that the edge is sufficient rounder, flattened, etc, that it will not readily cut tissue. In FIG. 15, the distal end 126′ is much more rounded than the second, inner needle 122 shown in FIGS. 7 and 8. Additionally, opposite the opening 130, the cannula 122′ has a flattened portion 132. The flattened portion 132 is configured to form part of a convex line between the projection 127 and the shorter distal end of the first, outer needle, without leaving so much of the second, inner needle 122 exposed that it would create crevices for bacteria of other microbes to become entrapped and carried into the blood stream, etc.

The distal end 118 of the first, outer needle 114 is shaped so as to leave a void 144 between the inner portion 120 of the distal end (i.e. the back side of the projection 127) and the distal end 126 of the second, inner needle or cannula 122′. This void 144 can be filled with a filler 146 which is preferably anti-microbial and/or has low adherence qualities for microbes, such as, for example, PLGA. The filler 146 is preferably used so as to create a substantially continuous distal surface 118b to the flattened back side 132 of the second, inner needle or cannula 122′. This provides a generally smooth plane along which the tissue through which the first, outer needle 114 passes can mechanically wipe any microbes off the distal end of the hypodermic needle system.

The rounded distal end 126 of the second, inner needle or cannula 122 allows the cannula to rotate with respect to the PLGA without requiring it to be torn or sheared. Additionally, the flattened portion 132 of the second, inner needle or cannula 122 allows the transition to be smooth as tissue passes over the distal end of the hypodermic needle system 110′, as the flattened portion is in general alignment with the opening in the distal end 18 of the first, inner needle 114.

When the first, outer needle 114 is in the desired location, the second, inner needle or cannula 122′ is rotated 180 degrees relative to the first, outer needle to expose the opening 130′ adjacent the distal end 126 of second, inner needle or cannula 122′. The opening may be, for example, 0.5 to 0.6 mm in diameter. Such a size easily aligns between the filler 146 and the short side of the distal end 118 of the first, outer needle 114.

One advantage of the configuration shown in FIG. 15 is that there is very little structure which could inadvertently snag on tissue as the hypodermic needle system 110′ is being withdrawn or if it is advanced further, even if in the open position where the opening 130′ is exposed.

Turning now to FIG. 16, there is shown a hypodermic needle system, generally indicated at 110′ such as may be used with the configuration shown in FIGS. 7-8. 15, etc. The needle system 110′ may include the first, outer needle 114 and the second, inner needle 122. The first, outer needle 114 may include an adaptor 250 disposed at or adjacent the proximal end of the first needle. The adaptor 25 may have one or more bases or flanges 260 is attached thereto which extend outwardly away from the needle

Likewise, the outer needle 122 may include an adaptor 250 which may be configured for attachment to a syringe such as is shown in prior figures. The adaptor 250 attached to the second, inner needle 122 may also be attached to one or more bases or flanges 264. A collar 258 may be used to space apart the adaptors 250 to hold the needles at the proper distance from one another.

The hypodermic needle system 110′ in FIG. 16 also includes a clip or engagement member 278 which engages or receives the flanges 260 and 264. While the clip 278 is in place, the flanges 260 and 264 are held in a rotational orientation relative to each other consistent with, for example, the second, inner needle 122 being held in the first, closed position (shown in FIG. 15) wherein the opening 130 in the second, inner needle is not exposed. Once the clip 278 is removed from engagement with at least one of the flanges 260 and 264, one of the flanges or bases can be rotated with respect to one another, to thereby move the second, inner needle 122 into the second, open position. This could be done, for example, by rotating one of the flanges 260 or 264 180 degrees about the associated needle, so thereby move the opening 130 in the second, inner needle into an open position. If configured as shown in FIG. 16, the clip could then be placed back on the flanges 260 and 264 to ensure that the needle is not inadvertently moved back to the original position. If such were to be done, the flanges 260, 264 may be color-coded or the like to make it clear when the two needles were in the first, closed position and the second, open position. (It will be appreciated that when one of the bases or flanges 260, 264 is pivoted about the needle so as to change the needles' relative orientations, that base or flange is acting as an actuator, as discussed above.)

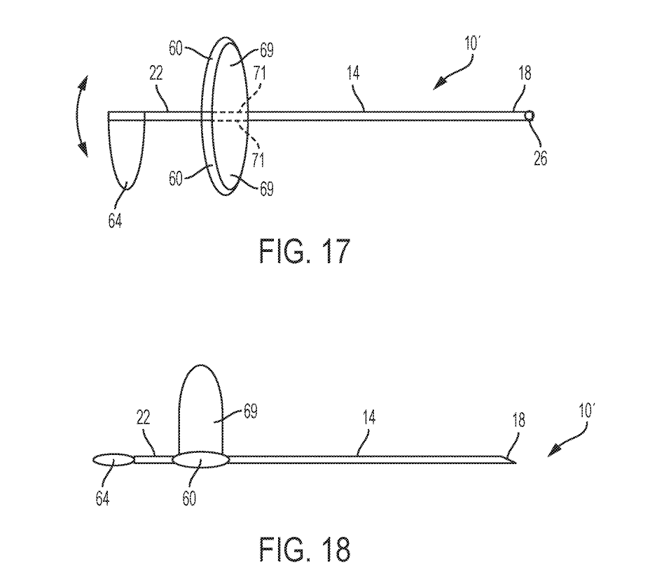

FIGS. 17 and 18 show a top view and a side view, respectively of a hypodermic needle system, generally indicated at 10′. The structure has several similarities with that shown in FIGS. 5 and 6 and is numbered accordingly. The hypodermic needle system 10′ includes the first, outer needle 14 and the second, inner needle 22. The distal end 18 of the first, outer needle 14 and the second, inner needle 22 can be aligned so as to be a first, closed position wherein the interior of the second, inner needle is not accessible through the open distal end of the first outer needle, and a second, open position wherein it is accessible.

In the embodiment shown in FIGS. 17 and 18, the orientation between the first, outer needle 14 and the second, inner needle 22 is regulated by a movement of an actuator 64 which is formed with or attached to the second, inner needle. Pivoting the actuator 64 moves the second, inner needle between the first, closed position and the second, open position in a similar manner as the actuator 64 in FIGS. 5 and 6. It will be appreciated that the second, inner needle 22 is usually connected to an adaptor or syringe for injecting or withdrawing fluid through the second, inner needle.

FIGS. 17 and 18 also show a base 60 which may be attached to the first, outer needle 14. The base can be used to help hold the hypodermic needle system 10′ in place. Also shown are a pair of support wings 69 which are disposed adjacent the base 60. The support wings 69 may have a score, cut-away, etc., which allows wings 69 to be bent upwardly as shown in FIG. 18. In this position the wings 69 provide a convenient handle whereby the hypodermic needle system 10′ may be held or withdrawn during use.

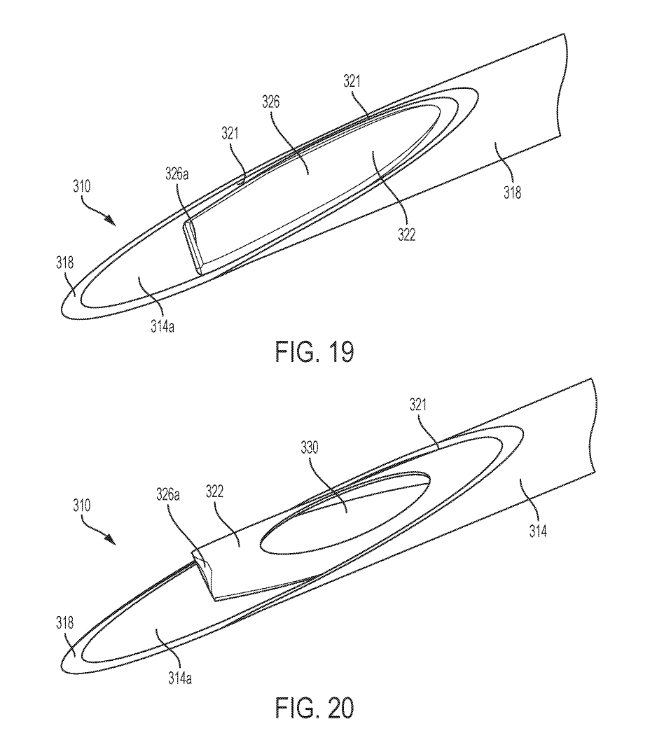

FIG. 19 shows a close-up view of another configuration of a hypodermic needle system 310 according to the present disclosure. The hypodermic needle system 310 includes a first, inner needle 314 and a second, inner needle 322 or cannula. It will be appreciated that any of the structures identified with respect to the prior configurations could be used with this configuration and vice-versa.

The first, outer needle 314 has a distal end 318 which is sharp for piercing the skin or other tissue. Unlike several of the prior configurations, the inner surface 314a of the first, outer needle 314 does not have a projection which mates with or is closely aligned with the distal tip 326a of second, inner needle 326. Rather a void is left which can be filled by a filling material, such as PLGA or other filers which one—reduce the risk of bacteria or other microbes being trapped in the void, and two—which is resistant to the bacteria or other microbes attaching to the filler material.

As will be apparent, there is also a seam 321 which runs along the interface between the first, outer needle 314 and the second, inner needle 318. The filler material can also be used to cover that seam 321, further reducing the risk of bacteria or other microbes being carried into the patient's blood stream, etc.

Once the needles of the needle system 310 are disposed in the desired location, the second, inner needle 326 can be rotated or advanced, as shown in FIG. 20, to expose the opening 330 through which medicine or other fluids may be injected, or through which blood or other body fluids may be withdrawn. Because the distal tip 326a is blunted, flattened, rounded, etc., there is little likelihood that the second, inner needle will lacerate blood vessels or other tissue. If will be appreciated that the inner needle could be coated with a release agent to assist it in moving against resistance provided by the filler/coating material.

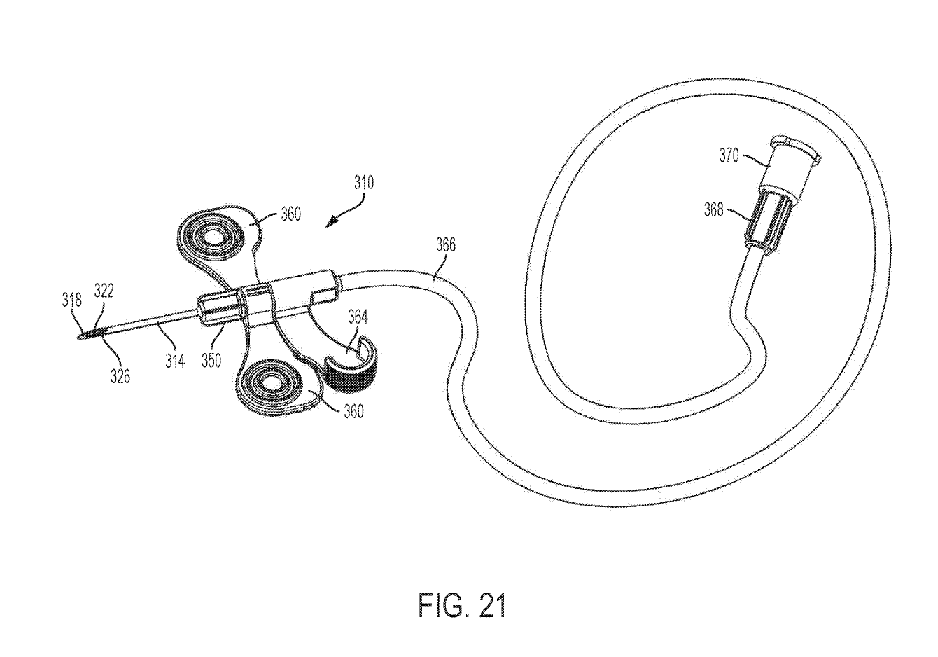

Turning now to FIG. 21, there is shown a perspective view of a hypodermic needle system, generally indicated at 310, as may be used with various configurations discussed herein. The needle system 310 may include the first, outer needle 314, and a second, inner needle 322 in the various configurations discussed above. The first, outer needle 314 may include a distal end 318 which is relatively sharp for piercing skin, etc., The second, inner needle 322 may have a distal end 322 which may be sharp or rounded and which may be covered, at least partially with a fill or sealing material.

The first, outer needle 314 may be attached to an adaptor 350 which holds the first, outer needle in place. The adaptor 350 may be connected to a base 360 which may have wings or projections to facilitate holding or attachment to the skin. Disposed adjacent the base 360 is an actuator 364. In configurations which rotate the second, inner needle 322, the adaptor 364 may be connected to the second, inner needle and pivotable with respect to the base 360, such that pivoting of the adaptor rotates the second, inner needle within the first, outer needle. In configurations in which the second, inner needle 322 is advanceable through the first, outer needle 314, the adaptor 364 may slide back and forth toward and away from the base 360.

The actuator 364 may be attached to tubing 366 at one end of the tubing, while a connector 368 and/or a luer-lock 370 may be attached at the other end of the tubing to facilitate the attachment of a syringe (not shown) or other container which can inject or receive fluid. Thus, it will be appreciated that the tubing 366 is typically disposed in fluid communication with the interior of the second, inner needle 322.

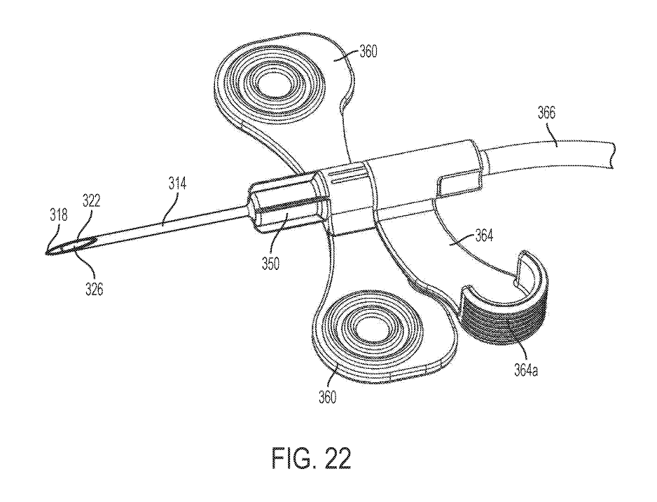

FIG. 22 shows a close-up view of the needle and actuation mechanism in the first, closed position. The actuator 364 includes a grip 364a which is disposed on the left side of the tubing 366 when viewed toward the distal. The second, inner needle 322 is rotated closed so that the distal end 326 closes the distal end 318 of the first, outer needle 314. In such a configuration, flow into the first, outer needle 314 and the second, inner needle is prevented.

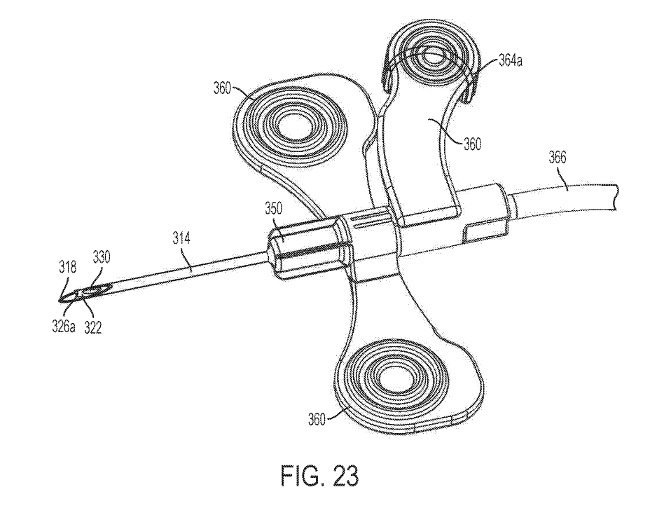

In contrast, FIG. 23 show a close-up view of the needle and actuation mechanism in a second, open position. The user has grabbed the grip or handle 364a and rotated the actuator 364 from the first position wherein it was on the left side of the tubing 366 when looking distally, to being generally on the right side of the tubing when viewed from the same orientation. Such a rotation causes the second, inner needle 322 to rotate thereby raising the distal tip 326a and exposing the opening 330 into the lumen defined by the second, inner needle. In such an orientation, medicine can be injected through the tubing 366 and through the second, inner needle 322 through the opening 330, or bodily fluids can be withdrawn through the opening, down the lumen of the needle and through the tubing. Because the distal end 326a of the second, inner needle 322 is rounded, blunted, etc., the risk of causing additional damage to a blood vessel is reduced. Additionally, as the distal end 118 may be covered with a filler material, the risk of bacteria being carried by the needles is substantially reduced.

Thus there is disclosed a contamination resistant hypodermic needle injection system and method of using the same. It will be appreciated that numerous modifications may be made without departing from the scope and spirit of this disclosure. The appended claims are intended to cover such modifications.

What is claimed is:

1. A hypodermic needle system comprising:
a first, outer needle having a bore and a distal end, the bore opening at the distal end; and
a second, inner needle disposed in the bore of the first, outer needle, the second inner needle having a closed end disposed in alignment with the distal end of the first, outer needle so as to occlude the distal end, and an opening proximal to the closed distal end, the first, outer needle and the second, inner needle being rotatable with respect to one another to move the opening from a first, closed position to a second, open position, the distal end of the second, inner needle leaving an area between the distal end of the second, inner needle and the distal end of the first, outer needle, and wherein the area is filled will a filler material.

2. The hypodermic needle system of claim 1, wherein the closed end of the second, inner needle is sloped to match a beveled face of the distal end of a first, outer needle, a seam being disposed at the juncture of the first, outer needle and the second inner needle, and wherein the seam is at least partially filled with a filler material.

3. The hypodermic needle system of claim 1, wherein the filler material comprises a bacterial resistant coating disposed on the distal end of the second, inner needle.

4. The hypodermic needle system of claim 1, wherein at least one of the first, outer needle and the second, inner needle is attached to an actuator.

5. The hypodermic needle system of claim 5, wherein the actuator is an arm.

6. The hypodermic needle system of claim 5, wherein the actuator comprises a threaded sleeve.

7. The hypodermic needle system of claim 5, wherein the actuator comprises a wheel.

8. The hypodermic needle system of claim 5, wherein the first, outer needle is attached to a base and the actuator is attached to the second, inner needle.

9. The hypodermic needle system of claim 1, wherein the closed end is rounded.

10. The hypodermic needle system of claim 1, wherein the second, inner needle has a distal tip which is blunted.

11. The hypodermic needle system of claim 1, wherein the second, inner needle has an adaptor for attachment to a syringe.

12. The hypodermic needle system of claim 1, wherein the second inner needle has a closed distal end and an opening adjacent to the closed distal end.

13. The hypodermic needle system of claim 1, wherein the second, inner needle has a flattened portion adjacent the closed distal end.

14. The hypodermic needle system of claim 1, wherein one or more bendable wings are connected to the first, outer needle.

15. The hypodermic needle system of claim 1, wherein the first, outer needle has a projection form on the inner surface thereof adjacent to the distal end.

16. A hypodermic needle system comprising:
a first, outer needle;
a second, inner needle disposed in the first, outer needle, the second, inner needle having a closed distal end and an opening proximal to the closed distal end, the second, inner needle being rotatable from a first, closed position wherein the opening is not exposed and a second, open position wherein the opening is exposed beyond the first, inner needle; and
wherein the second, inner needle has a distal tip which is rounded, blunted or flattened.

17. The hypodermic needle system of claim 16, further comprising a base attached to one of the first, outer needle and the second, inner needle.

18. The hypodermic needle system of claim 17, further comprising an actuator and wherein moving the actuator toward the base moves the closed distal end of the second, inner needle to thereby uncover the opening.

19. The hypodermic needle system of claim 17, further comprising an actuator and wherein pivoting the actuator about one of the first, outer needle and the second, inner needle moves hypodermic needle system between a first, closed position and a second, open position.

20. The hypodermic needle system of claim 16, further comprising an actuator attached the second, inner needle.

21. The hypodermic needle system of claim 16 wherein the second, inner needle has an adaptor for attaching a syringe thereto.

22. The hypodermic needle system of claim 16, wherein the closed end of the second, inner needle has a rounded distal end.

23. A hypodermic needle system comprising:
a first, outer needle having a distal end;
a second, inner needle disposed in the first, outer needle, the second, inner needle having a closed distal end which is rounded, flattened or blunted at its distal tip second, inner needle being advanceable from a first, closed position wherein the opening is not exposed and a second, open position wherein the opening is exposed beyond the first, inner needle; and
a filler material disposed between the distal tip of the second, inner needle and the distal end of the first, outer needle.

24. The hypodermic needle system of claim 23, further comprising a base attached to one of the first, outer needle and the second, inner needle.

25. The hypodermic needle system of claim 24, further comprising an actuator and wherein moving the actuator toward the base moves the closed distal end of the second, inner needle to thereby uncover the opening.

26. A method for injecting or withdrawing fluid from a patient, the method comprising:
selecting a hypodermic needle system having a first, outer needle and a second, inner needle, the second inner needle having a closed distal end and an opening proximal to the closed distal end, the needles being movable with respect to one another between a first, closed position wherein the first, outer needle occludes the opening and a second, open position wherein the first, outer needle does not occlude the opening of at least one of the first, outer needle and the second inner needle having a bacteria resistant coating disposed thereon;
inserting the hypodermic needle system while in the first, closed position; and
moving at least one of the first, outer needle and the second, inner needle to move the hypodermic needle system into the second, open position; and
passing a fluid through the second, inner needle.

27. The method according to claim 26, wherein the bacteria resistant coating is disposed on the distal end of the first, outer needle and the second, inner needle, and wherein the method comprises breaking the coating when moving the hypodermic needle system into the second, open position.

28. The method according to claim 26, wherein moving at least one of the needles so move the hypodermic needle system in to the second, open position comprises moving an actuator attached to the second, inner needle.

29. The method according to claim 268, wherein a base is attached to the first, outer needle and the method comprises rotating the actuator with respect to the base.

30. The method according to claim 28, wherein a base in attached to the first, outer needle and the method comprises sliding the actuator with respect to the base.

31. The method according to claim 28, wherein the hypodermic needle is inserted in the patient and wherein moving the actuator is performed with one hand.

32. The method according to claim 26, wherein the method comprises pivoting an actuator about the first, outer needle to rotate the first, outer needle and thereby expose an opening in the second, inner needle.

33. The method according to claim 26, wherein the method comprises pivoting an actuator about the second, inner needle to rotate the second, inner needle and thereby expose an opening in the second, inner needle to be in alignment with an opening in the distal end of the first, outer needle.
